(19) United States
(12) Patent Application Publication
(10) Pub. No.: US 2005/0112551 A1
(43) Pub. Date: May 26, 2005

(54) Dual assay for evaluating activity and cytotoxicity of compounds in the same population of cells

(75) Inventors: Wade Stanton Blair, San Marcos, CA (US); Joan Qun Cao, Carlsbad, CA (US); Jason Isaacson, San Diego, CA (US); Amy Karen Patick, Escondido, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/721,405

(22) Filed: Nov. 24, 2003

(51) Int. Cl. C12Q 1/70; C12Q 1/68; C12N 15/867; C12N 15/87

(52) U.S. Cl. 435/5; 435/6; 435/455; 435/456

(57) ABSTRACT

Methods are provided for evaluating the activity and cytoxicity of a compound in the same population of cells. These dual activity/cytoxicity methods are amenable for use in a high-throughput format. Also provided are humanized Renilla luciferase genes useful for the dual activity/cytoxicity assays and for use in a variety of reporter constructs.

### FIELD OF THE INVENTION

The present invention is directed generally to a dual activity/cytotoxicity assay that allows for the evaluation of the activity and cytotoxicity of compounds in the same population of cells. The assay is also amenable to a high-throughput format. More specifically, the invention relates to a rapid and highly quantitative method for evaluating the activities and cytotoxicities of drugs in dose response assays, in the same population of cells. The present invention also relates generally to the field of reporter genes useful for reporter assays, including the dual activity/cytoxicity assays of the invention. In particular, the invention relates to improved Renilla reniformis luciferase (rluc) genes, constructs and methods of use. The luciferase genes disclosed herein are humanized Renilla reneformis luc genes adapted for expression in mammalian cells through use of codon sequences optimized for expression in mammalian cells.

### BACKGROUND OF THE INVENTION

Reporter genes are used throughout the biological sciences as a means to identify and analyze regulatory elements of genes. Using recombinant DNA techniques, reporter genes can be fused to a regulatory sequence of interest. The resulting recombinant is then introduced into cells where the expression of the reporter can be detected using various methods, including measurement of the reporter mRNA, measurement of the reporter protein, or measurement of the reporter enzymatic activity. Commonly used reporter genes include beta-galactosidase, firefly luciferase, bacterial luciferase, Renilla luciferase, alkaline phosphatase, chloramphenicol acetyltransferase (CAT), green fluorescent protein (GFP) and beta-glucuronidase (GUS).

Many reporter systems utilize luciferase genes. Luciferase refers to a group of enzymes that catalyze the oxidation of various substrates to produce a light emission. Generally, luciferase activity is not found in eukaryotic cells. Thus, it is advantageous for studying promoter activity in mammalian cells. The most popular luciferases for use as reporter genes are the bacterial luciferases, the firefly (Photinus pyralis) luciferase, the Aequorin luciferase and more recently the Renilla luciferase. The different luciferases have different specific requirements and may be used to detect and quantify a variety of substances. For example, one major application for the use of the firefly luciferase is to detect the presence of ATP. The purified jellyfish photoprotein, aequorin, is used to detect and quantify intracellular Ca²⁺.

The wild-type luciferase enzyme of the sea pansy Renilla reniformis is a monomeric protein with a molecular weight of 36 kDa. This enzyme catalyzes the emission of visible light in the presence of oxygen and the luciferin coelenterazine to produce blue light. The luciferase gene from Renilla has been used to assay gene expression in bacterial (Jubin et al., Biotechniques 24:185-188 (1998)), yeast (Srikantha et al., J. Bacteriol. 178:121-129 (1996)), plant (Mayerhofer et al., Plant J. 7:1031-1038 (1995)), and mammalian cells (Lorenz et al., J. Biolumin. Chemilumin. 11:31-37 (1996)).

The cloning, expression and use of wild-type Renilla luciferase are reported in U.S. Pat. Nos. 5,292,658 and 5,418,155.

Firefly luciferase and Renilla luciferase are available commercially (Boehringer Mannheim, Sigma, and Promega). Promega has developed a synthetic Renilla luciferase gene that contains codons optimized for efficient expression in mammalian cells. Literature from Promega indicates that additional features of this modified gene include removal of potentially interfering restriction sites and genetic regulatory sites from the gene (Promega Technical Manual No. 055, revised June 2001). Sequence information related to various plasmids containing the Promega humanized Renilla luciferase gene are deposited with GenBank under accession numbers AF362545-AF362551.

Other examples of genes and reporter genes optimized for expression in mammalian cells are known in the art. For example, Seed et al. report a method for increasing the expression of eukaryotic and viral genes in eukaryotic cells that involves replacing non-preferred amino acid codons with preferred codons that encode the same amino acid (U.S. Pat. No. 6,114,148; Haas et al., Current Biology 6:315-323 (1996)) (both incorporated herein by reference). Muzyczka et al. (U.S. Pat. No. 6,020,192) and Zolotukhin, et al. (J. Virology 70:4646-4654 (1996)) report green fluorescent proteins optimized for expression in mammalian cells. Sherf et al. report a modified beetle luciferase (U.S. Pat. No. 5,670,356).

Reporter genes have been used to monitor the expression of genes from pathogenic organisms that are differentially expressed after a pathogen invades a host cell. For example, promoter GFP fusions were used to monitor expression from two genes of Legionella pneumohilai in Acanthamoeba castellar cells (Kohler et al., Mol. Gen. Genet 262:1060-69 (2000)), and a library of Mycobacterium marinum promoter-GFP fusions were used to identify Mycobacterium genes expressed in macrophages (Barker et al., Mol. Microbiol. 29:1167-77 (1998)). Reporter gene technologies have also been used to study gene expression and intracellular survival in Yersinia pestis (Pollack et al., Nature 322:834-836 (1986); Forsberg et al. Infect. Agents Dis. 2:275-278 (1993)); Yersinia enterocolitica (Jacobi et al., Mol. Microbiol. 30(4):865-882 1998)); Salmonella typhimurium (Garcia del Partillo et al., Mol Microbiol. 6:3289-3297 (1992)), Bartonella (Schulein et al., J. Exp. Med. 193:1077-1086 (2001)), Staphylococcus aureus (Qazi et al., Infect. Immun. 69:7074-82 (2001)), Legionella (Kohler et al., supra), and Leishmania spp. (Roy et al., Mol. Biochem. Parasitol. 110:195-206 (2000)).

Reporter genes have also been used to monitor the activity of cell-receptors (both external and intracellular) and signaling pathways (Naylor, Biochemical Pharmacol. 58:749-757 (1999)).

Reporter genes have been used in assays for drug discovery. For example, recombinant cells that express cell surface receptors and that contain reporter-gene constructs responsive to the activity of the cell-surface receptor have been reported for the use of identifying agonists and antagonists of such receptors (see, e.g., U.S. Pat. Nos. 5,401,629; 5,436,128; 5,922,549; and 6,159,705).

Multiple assay formats are a known tool for evaluating the potential antiviral activity of putative inhibitors. Common antiviral assay methods include quantitatively measuring the production of viral antigens (e.g., HIV-1 p24) or the activities of viral enzymes (e.g., HIV-1 reverse transcriptase) as indicators of virus replication. Although highly sensitive, these methods are often cumbersome and difficult to format for high-throughput screening. Alternatively, virus replication can be measured indirectly by monitoring viral-induced host-cell cytopathic effects using dye reduction methods (cell protection assays), which are simple and can usually be adapted for medium- to high-throughput analyses. However, cell-protection assays are limited to highly lytic virus replication systems and often require lengthy assay timeframes (>4 days).

Reporter-virus systems have been described for many different viruses, including human immunodeficiency virus type 1 (HIV-1) (Terwilliger et al., Proc. Natl. Acad. Sci. 86:3857-3861 (1989); Chen et al., J. Virol. 68(2):654-660 (1994)), human herpes viruses (Arthur et al., J. Virol. 75(8):3885-3895 (2001); Van Den Pol et al., J. Neurosci. 19(24):10948-10965 (1999)), papillomavirus (Rossi et al., Hum. Gene Ther. 11(8):1165-1176 (2000)), picornaviruses (Percy et al., J. Virol. 66(8):5040-5046 (1992); McKnight et al. J. Virol. 70(3):1941-1952 (1996)), rhabdoviruses (Boritz et al., J. Virol. 73(8):6937-6945 (1999)), alphaviruses (Olkkonen et al., Methods Cell Biol. 43(Pt A):43-53 (1994); Dubensky et al., J. Virol. 70(1):508-519 (1996)), influenza virus (Neumann et al., J. Virol. 74(1):547-551 (2000)), flaviviruses (Khromykh et al., J. Virol. 71(2):1497-1505 (1997)), pestiviruses (Tautz et al., J. Virol. 73(11):9422-9432 (1999)), and hepatitis C virus (HCV) (Krieger et al., J. Virol. 75(10):4614-4624 (2001)).

Depending on the reporter gene utilized, reporter virus assays are often highly sensitive and amenable to a high-throughput format. However, the reporter virus approach is limited to either (1) viruses for which an infectious or replication competent viral cDNA is available or (2) viruses for which systems to introduce foreign genes through homologous recombination in infected cells are available. An alternative approach utilizes an indicator cell. In such assays, virus replication is indirectly measured by monitoring the induction of a reporter gene under the control of a viral promoter present in the target cell (Kimpton et al., J. Virol. 66(4):2232-2239 (1992); Kung et al., J. Virol. Methods. 90(2):205-212 (2000)). As is the case with reporter virus systems, indicator-cell systems can be adapted to a high-throughput format.

One component of a cell-based drug-screening assay is often the assessment of a test compound's cytotoxicity or specificity. Cytotoxicity measurements when combined with compound activity data allow the distinction of specific compound activity from non-specific inhibitor effects or cytotoxicity. In fact, for the majority of the assays mentioned, including reporter virus assays, a separate assay format must be used to evaluate inhibitor-mediated cytotoxicity. In cell protection assays, antiviral and cytotoxic effects of an inhibitor can be measured using the same method; however, accurate evaluations of antiviral and cytotoxic activities must be performed in separate assays (i.e., separate cell populations). Therefore, antiviral screens using the current assay formats must include a separate counterscreen to evaluate a compound's cytotoxicity, which can lead to a significant increase in resource requirements and a significant reduction in ultimate inhibitor identification rate (i.e., overall screen throughput).

Reporter gene-based cytotoxicity assays that exhibit potential high-throughput capabilities have been reported (Memon et al., J. Immunol. Methods 180(1):15-24 (1995); Schafer et al., J. Immunol. Methods 204(1):89-98 (1997); Sandman et al., Chem. Biol. 6(8):541-551 (1999)). In one assay format, a reporter gene was expressed in cells and the effect of compounds on cell viability (Sandman et al., 1999, supra) or programmed cell death (Memon et al., 1995, supra) was monitored by measuring intracellular expression of the reporter gene in the compound-treated cells. In a second format, a reporter gene was expressed in cells, and cell death was measured by quantifying the release of the reporter enzyme into the supernatant following cell lysis (Schafer et al., 1997, supra). In addition to simple cytotoxicity assays, reporter systems have been reported that distinguish specific inhibitors of HIV-1 LTR gene expression from non-specific inhibitors or cytotoxic agents (Watson et al. AIDS Res. Hum. Retroviruses 9(9):861-867 (1993); Del Rosario et al., Nat. Biotechnol. 14(11):1592-1596 (1996)).

A commercially available reporter system (Dual-Luciferase® Reporter Assay System) is available from Promega Corporation that first measures firefly luciferase activity followed by measurement of Renilla luciferase activity (see U.S. Pat. No. 6,171,809). Dual measurement of firefly and Renilla luciferase activity in transfected cells is described in U.S. Pat. Nos. 6,261,791, 6,235,873, 6,255,112, 6,255,473, 6,143,502 and 6,063,578. The Promega single and dual reporter assays based on luciferase activity are also discussed in International Publication No. WO 96/40988.

### SUMMARY OF THE INVENTION

Described herein are methods for evaluating both activity and cytotoxicity of test compounds in the same population of cells. The methods involve the steps of providing a target cell that contains a first reporter gene that monitors cell viability. To these cells are added a test compound. After addition of the test compound, a second reporter gene is introduced into the cells, wherein the second reporter gene is different than the first reporter gene. The second reporter gene is indicative of the activity of a microorganism or cell receptor and monitors the activity of the test compound. After expression of the reporter genes, the activities of both reporters are measured and compared to the activity of the same reporter genes measured in the absence of a test compound.

Another dual activity/cytoxicity assay of the invention involves providing target cells that contain a first reporter gene that monitors cell viability, and a second reporter gene that is indicative of the activity of a microorganism or cell receptor. The second reporter gene is different than the first reporter gene. To these cells are added a test compound. After expression of the reporter genes, the activities of both reporters are measured and compared to the activity of the same reporter genes measured in the absence of a test compound.

All of the dual activity/cytotoxicity assays described herein are amenable to a high-throughput format.

The second reporter gene used in the assays can be indicative of the activity of intracellular pathogens. For example, the second reporter gene can monitor the activity of the following pathogens: human immunodeficiency virus, hepatitis A virus, hepatitis B virus, hepatitis C virus, herpes simplex virus, human herpes virus, human rhinovirus, picornavirus, influenza virus, rhabdovirus, papilloma virus, Salmonella, Shigella, Yersinia, Mycobacterium, Listeria, Stapylococcus, Chlamydiae, Legionella, or Leishmania.

The second reporter gene can also be indicative of the activity of a cell receptor, such as a kinase receptor, G protein-coupled receptor, or ion-channel receptor.

Possible combinations of first and second reporters include: Photinus pyralis (firefly) luciferase and Renilla luciferase; firefly luciferase and secreted alkaline phosphatase; Renilla luciferase and secreted alkaline phosphatase; firefly luciferase and Aequora victoria green fluorescent protein; Renilla luciferase and Aequora victoria green fluorescent protein; or beta-galacosidase and firefly luciferase.

Preferred reporter genes for use in the assays described herein are humanized Renilla reneformis luciferase genes. Particularly preferred are humanized Renilla luciferase nucleic acid molecules that encode functional luciferase polypeptides, wherein such nucleic acid molecules contain humanized codons not found in wild type Renilla reniformis luciferase. Such nucleic acid molecules will contain at least 15% of the humanized codons shown in the nucleic acid sequence of SEQ ID NO:1 (FIG. 5).

Reporter constructs that monitor a pathogen's activity can be constructed in any organism that infects mammalian cells. For example, a reporter virus can be constructed by inserting a reporter gene in a viral region non-essential for viral replication. Alternatively, reporter genes that indirectly monitor a pathogen's activity can be constructed in any target cell. For example, a target cell can be constructed that contains a reporter gene, the expression of which is under the control of a promoter element that is activated upon infection of the target cell by the pathogen.

Preferred viral reporter constructs contain a humanized Renilla luciferase nucleic acid molecule that encodes a functional luciferase polypeptide. The nucleic acid molecule contains humanized codons not found in wild type Renilla reniformis luciferase, and said humanized codons comprises at least 15% of the humanized codons shown in the nucleic acid sequence of SEQ ID NO:1.

The present invention is also directed to humanized nucleic acid molecules that encode functional Renilla luciferase polypeptides. Such nucleic acid molecules contain humanized codons not found in wild type Renilla reniformis luciferase, and the humanized codons comprise at least 15% of the humanized codons shown in the nucleic acid sequence of SEQ ID NO:1.

Another preferred nucleic acid molecule is represented by the sequence of SEQ ID NO:1.

The nucleic acid molecules of the invention can be operably linked to a promoter region, or attached to a heterologous nucleic acid molecule. The heterologous nucleic acid can encode a fusion protein or fusion peptide.

The nucleic acid molecules of the invention can be used in vector constructs. Preferred constructs are viral reporter constructs, such as human immunodeficiency virus, hepatitis A virus, hepatitis B virus, hepatitis C virus, herpes simplex virus, human rhinovirus, picornavirus, influenza virus, rhabdovirus, or papilloma virus.

In another embodiment of the invention host cells contain a nucleic acid molecule of the invention. Examples of suitable host cells are HeLa cells, VERO cells, CHO cells, COS cells, BHK cells, HEPG2 cells, 3T3 cells, or 293 cells.

The nucleic acid molecules of the invention may also be used in a kit.

Other aspects, features, and advantages of the invention will be become apparent upon consideration of the detailed description below in conjunction with the appended figures.

### BRIEF DESCRIPTION OF THE DRAWINGS

FIG. 1 is a schematic diagram showing an HIV dual activity/cytotoxicity reporter assay where HeLa HRLuc target cells are infected with VSV/HIVLuc in the presence or absence of a compound.

FIG. 2 shows interplate reproducibility of antiviral and cytotoxicity measurements for test compounds added to the HeLa HRLuc target cells and infected with VSV/HIVLuc as shown in FIG. 1.

FIG. 3 shows the distribution of well activity in a mock screen. The percent inhibition values calculated for the antiviral and cytotoxicity measurements in different wells where test compounds were added to target cells and infected with VSV/HIVLuc as shown in FIG. 1 are plotted versus the number of wells with that activity.

FIG. 4 shows HRLuc versus RLuc expression in transfected cells.

FIG. 5 shows the nucleotide sequence of HRLuc as compared to wild type Renilla luciferase.

### DETAILED DESCRIPTION OF INVENTION AND ITS PREFERRED EMBODIMENTS

A. Dual Activity/Cytotoxicity Reporter Assays

The present invention is directed to a dual activity/cytotoxicity reporter assay useful for evaluating the activity and cytotoxicity of potential drugs (e.g., antimicrobials and modulators of cell-receptor activity) in the same population of cells. Such assays are amenable for use in high-throughput formats. In addition, the present invention is directed to assay systems that allow for a rapid and highly quantitative evaluation of the activities and cytotoxicities of compounds in a dose response assay.

As used herein, the terms “comprising” and “including” are used in an open, non-limiting sense.

The term “in the same population of cells” means that the assays described herein are performed on cells from the same vessel. By vessel is meant a well, e.g., as found in a microtiter plate, or test tube.

The dual activity/cytotoxicity reporter assay system of the invention can be used for compound screening in any system in which compound activity can be monitored through use of a reporter construct (compound-reporter) and cell viability can be measured through use of a different reporter construct. Compound-reporter constructs can be made in pathogens known to infect mammalian cells, such as viruses, bacteria, fungi and protozoa.

Alternatively, compound-reporter constructs can be made by introducing pathogen responsive reporters into target cells. In this case, both the compound-reporter and the cytotoxicity reporter are introduced into the target cell prior to infection with a pathogen. Expression of the compound-reporter is under the control of a promoter element that is responsive to infection of the target cell by a pathogen. As before, compound activity is measured by monitoring expression of the compound-reporter. The cytotoxicity reporter, which utilizes a reporter gene different from the compound-reporter, is constitutively expressed in the target cell and is used to measure cell viability.

The dual activity/cytotoxicity assay can also be used to screen for compounds that modulate cell receptor activity (both intracellular and extracellular receptors), in which a reporter construct that contains an element responsive to the activity of the cell receptor is available. Such cell receptor constructs include reporter constructs responsive to the activity of kinase receptors, G protein-coupled receptors, and ion channels.

In the dual activity/cytotoxicity reporter assay system of the invention, target cells can be constructed that constitutively express a reporter gene responsive to cell viability. As a result, the reporter gene is expressed as long as the target cell remains viable and transcriptionally active. Thus, the reporter is a monitor of cytotoxicity.

The reporter responsive to cell viability may be integrated into the genome of the target cell or it may be contained on an extrachromosmal element, such as a vector. After construction of the target cell, the cell is transduced or transfected with the reporter construct for which drug discovery or development or drug testing is sought, for example, a viral reporter construct. This construct would encode a reporter gene different from that already present in the target cell. After expression, the activity of the reporter genes is measured, in the presence or absence of a compound of interest, using a dual reporter assay method which allows for the measurement of multiple reporter genes in the same population of infected cells, i.e., in the same well in a microtiter assay plate.

For example, Applicants have constructed a target cell line that constitutively expresses a humanized Renilla luciferase gene (HeLa HRLuc, constructed as described herein). These cells were used to evaluate the antiviral activities and cytotoxicities of HIV-1 reverse transcriptase (RT) and integrase (IN) inhibitors. Half-log dilutions of test compounds were added to HeLa HRLuc target cells. Compound-treated or compound-free target cells were then infected with an HIV-1 reporter virus (VSV/HIVLuc, described herein). Seventy-two hours after infection, the activities of firefly luciferase (the viral-encoded reporter) and Renilla luciferase (the target cell-encoded reporter) were measured in the infected cells using a dual-luciferase reporter assay system (Promega). As shown in Table 1, the antiviral activities (EC₅₀) and cytotoxicities (CC₅₀) of the non-nucleoside reverse transcriptase inhibitors (NNRTIs) (CPV, DLV, EFV), nucleoside reverse transcriptase inhibitors (NRTIs) (AZT, DDI, 3TC, and D4T), and IN inhibitors (L-731988 and L-798906) tested in the dual activity/cytoxicity assay were similar to those observed in cell-protection assays using HIV-1 RF and CEM-SS cells.

The introduction of the compound-reporter should not have a significant effect on cytoxicity. Significant effect on cytotoxicity is defined as a >90% cytotoxic effect on target cells in the absence of compound. For example, the dual activity/cytotoxicity assay when used with reporter viruses combines short assay time frames with either non-cytopathic reporter viruses or low reporter virus inputs such that cytopathic effects are not significant. Alternatively, the dual activity/cytotoxicity assay when used in a co-culture system, where one cell type that supports cytopathic replication of virus is co-cultured with a second cell type which serves a the indicator for activity/cytotoxicity. As a result, compound antiviral and cytotoxic activities can be accurately measured in the same well or population of infected cells. Likewise if a reporter system is made in other pathogenic microorganisms, introduction of the microorganism should not cause significant cytopathic effects within the time frame of the assay.

TABLE 1
Antiviral Activities of RT and IN Inhibitors in the Dual Reporter Assay

| | | | | HIV-1 RF Cell | | |
|---|---|---|---|---|---|---|
| | Dual Reporter Assay^{a} | | | Protection Assay^{b} | | |
| Com- | EC₅₀ | CC₅₀ | | EC₅₀ | CC₅₀ | |
| pound | (uM) | (uM) | TI^{c} | (uM) | (uM) | TI |
| CPV | 0.0015 | 36.4 | 24,266 | 0.0014 | 66.1 | 47,214 |
| NVP | 0.031 | >32 | >1032 | 0.033 | >1000 | >30,303 |
| DLV | 0.020 | >100 | >5000 | 0.013 | 90.5 | 6,961 |
| EFV | 0.0004 | 71.7 | 179,250 | 0.0015 | 57.4 | 38,266 |
| AZT | 0.029 | >320 | >11,034 | 0.008 | >320 | >40,000 |
| 3TC | 0.14 | >32 | >229 | 0.065 | >1000 | >15,384 |
| d4T | 0.27 | >32 | >119 | 0.51 | >32 | >64 |
| ddl | 0.59 | >32 | >54 | 12.9 | >32 | >10 |
| L-798906 | 3.2 | 129 | 40.3 | 1.5 | 150 | 100 |
| L-731988 | 1.8 | 166 | 92.2 | 4.5 | 250 | 55.6 |

^{a}Antiviral activity determined using the dual reporter assay ˜72 hours after VSV/HIVLuc infection of HeLa HRLuc cells. Results represent the mean of 2 to 3 experiments.
^{b}Antiviral activity determined by measuring XTT dye reduction six days after infection of CEM-SS cells with HIV-1 RF infection. Results represent the mean of 2 to 3 experiments.
The therapeutic index (TI) was calculated by dividing the cytotoxicity (CC₅₀) by the antiviral activity (EC₅₀).

In another embodiment of the invention, the dual activity/cytotoxicity reporter assay system can be used to determine the specific antiviral activity of inhibitors in standard dose response assays. In such assays, target cells are infected with reporter virus in microtiter (e.g., 96 well) plates containing serial dilutions of test inhibitors or no inhibitor. At a specified time after infection, the activities of the viral and target cell-encoded reporter genes are measured in the infected cells using the appropriate dual reporter assay methods. Data from the reporter gene measurements can be expressed as the percent of reporter gene activity in infected inhibitor-treated cells relative to that of infected, inhibitor-free cells. An analysis of the antiviral component of such data allows for the calculation of the fifty-percent effective concentration (EC₅₀) or ninety-percent effective concentration (EC₉₀) of an inhibitor. In addition, an analysis of the cytotoxicity component of the data can be used to calculate the 50% cytotoxicity concentration (CC₅₀) of an inhibitor. The therapeutic index (TI), which is a measurement of the specific antiviral activity of an inhibitor, can then be calculated by dividing the cytotoxicity (CC₅₀) by the antiviral activity (EC₅₀).

The order of steps in the dual activity/cytotoxicity reporter assays of the invention can determine what types of compounds one would isolate as potential drugs. For example, by introducing the compound reporter construct, such as a reporter virus, to the target cells after adding the compounds to be tested, one may isolate compounds that interfere with entry or attachment of a pathogen, such as a bacteria or virus. If the compound reporter construct is introduced into the target cells before the compounds to be tested, then compounds that prevent entry or binding of a pathogen to a target cell may not be isolated.

The dual activity/cytotoxicity reporter assay system can be used to screen for specific antiviral inhibitors in a high-throughput format. In a high-throughput format, putative inhibitors are added at single or multiple doses to target cells in microtiter plates. Reporter virus are then added to the wells containing target cells and inhibitor or no inhibitor control wells. At a specified time after infection, the activities of the viral and target cell-encoded reporter genes are measured in the infected cells using the appropriate dual assay methods. Data from the reporter gene measurements can then be expressed as the percent inhibition of reporter gene activity in infected inhibitor-treated cells relative to that of infected inhibitor-free cells. Antiviral activity is then assigned to test inhibitors that (1) effect a significant reduction in the viral-encoded reporter gene activity relative to the no compound control wells and (2) show no significant effect on expression of the target cell-encoded reporter gene relative to the no compound control wells.

A significant reduction in reporter gene activity in a screening format is defined by the practitioner of the assay and can be based on any one or more of the following: (1) assay reproducibility, (2) desired hit rate, (3) hit confirmation rate, and/or (4) compound library properties. Therefore, reductions in reporter gene activity determined to be significant may vary depending on the properties of the assay and the goals of the screen. A typical range can be anywhere from 30% to 90% reduction.

In another embodiment of the invention, activity/cytotoxicity indicator cells could be utilized to determine the antiviral activities and cytotoxicities of compounds. The activity/cytotoxicity indicator cells are cells that (1) contain a reporter gene under the control of a promoter element that is responsive to viral infection of the target cell and (2) constitutively express a different reporter gene. Compound activity is measured by monitoring expression of the viral infection-responsive reporter gene (compound-reporter), while cytotoxicity is measured by monitoring expression of the reporter gene constitutively expressed in the target cell. To evaluate the antiviral activities and cytotoxicities of compounds using this assay format, the indicator cells are infected with virus or co-cultured with virus-infected cells in microtiter plates containing test inhibitors or no inhibitor. At a specified after infection, the activities of the compound-reporter and cytotoxicity reporter are measured using the appropriate dual reporter assay methods.

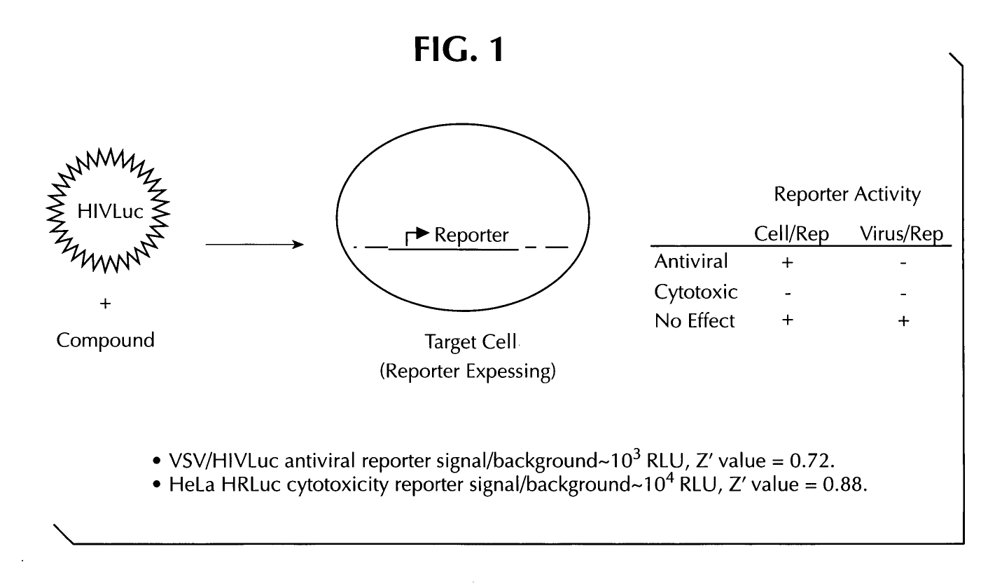

To show that the dual activity/cytotoxicity reporter assays of the invention are amenable for identifying potential drugs in a high-throughput format, coefficients of variation and screening window coefficients (Z′ value) were calculated for the following assay. A test compound or DMSO was added to HeLa HRLuc target cells (HeLa cells which contain a constitutively expressed humanized Renilla luciferase gene, described herein), seeded in 96-well plates. These cells were then infected with a reporter virus construct (VSV/HIVLuc, an HIV reporter virus that contains the firefly luciferase gene, described herein). Seventy-two hours after infection the activities of firefly luciferase (the viral-encoded reporter) and Renilla luciferase (the target cell-encoded reporter) were measured. Data from the reporter gene measurements were expressed as the percent inhibition of reporter gene activity in infected compound-treated cells relative to that of infected compound-free cells. As shown in FIG. 1, the antiviral and cytotoxicity components of the assay exhibited coefficients of variation (CV) of 19.5% and 13.4%, respectively, and Z′ values of 0.72 and 0.88, respectively. The Z′ value is reflective of the dynamic range as well as the variation of the assay and is a useful tool for assay comparisons and assay quality determinations (Zhang et al., J. Biomolec. Screen 4:67-73 (1999)). Typically a Z′ value >0.5 is considered favorable for high-throughput screening. Therefore, the low CVs and favorable Z′ values suggest that the dual activity/cytotoxicity reporter assay is suitable for high-throughput screening.

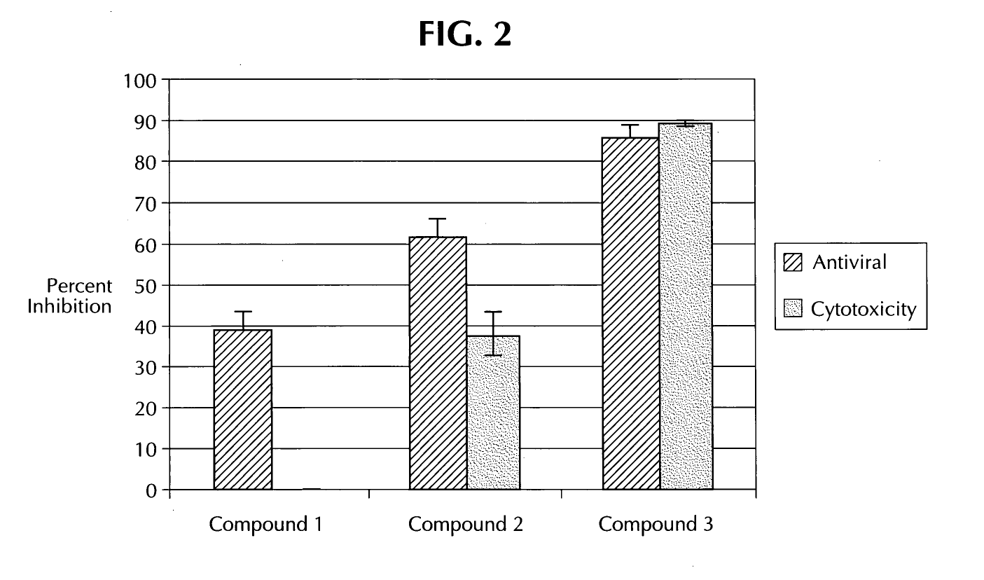

To further demonstrate that the dual activity/cytotoxicity reporter assay is suitable for high-throughput screening, 96-well mock screening plates were evaluated using the VSV/HIVLuc reporter virus and the HeLa HRLuc cell line. Test compound or DMSO alone was added to HeLa HRLuc target cells, and the compound or DMSO-treated HeLa HRLuc target cells were then infected with VSV/HIVLuc. Seventy-two hours after infection, the activities of firefly luciferase (the viral-encoded reporter) and Renilla luciferase (the target cell-encoded reporter) were measured in the infected cells using a dual reporter assay. Data from the reporter gene measurements were expressed as the percent inhibition of reporter gene activity in infected compound-treated cells relative to that of infected compound-free cells. For the experiment reflected in FIG. 2, each of the three compounds was introduced into five wells distributed randomly across five different plates, resulting in a total of 10 mock plates. Compound 1 was introduced at the predicted antiviral 50% effective concentration (EC₅₀) for that compound. Compound 2, which exhibited both antiviral activity and subtle cytotoxicity, was introduced at a concentration corresponding to its predicted antiviral 60% effective concentration and 25% cytotoxic concentration, while compound 3 was introduced at the predicted 80% cytotoxic concentration. The data show a highly reproducible measurement of the antiviral activities and/or cytotoxicities of the test compounds across microtiter plates (interplate standard deviation <14%) (FIG. 2). In addition, the levels of reporter gene inhibition observed for the antiviral and cytotoxicity components of the assay were similar to that expected for all three compounds. Therefore, these data demonstrate the reproducible identification of specific antiviral compounds using the dual activity/cytotoxicity assay in a high-throughput format.

The dual activity/cytoxicity reporter assay system of this invention can be used for any virus for which an infectious and/or replication competent molecular clone is available and can be used to generate a reporter virus. As used herein, “infectious and/or replication competent molecular clone” means any cDNA containing viral sequence that encodes the potential to direct replication of either full-length or subgenomic viral cDNAs via viral promoters and/or replication signal sequences. In addition, the dual activity/cytotoxicity reporter assay system can be used for any reporter virus generated through homologous recombination events in an infected cell. Furthermore, the dual activity/cytotoxicity reporter assay system can used for any pathogen that generates a response in the target cell that is dependent on infection of the target cell and that can be measured using a reporter gene. For example, the dual activity/cytotoxicity reporter assay would be suitable for analyzing human immunodeficiency viruses (HIV-1 and HIV-2), hepatitis A virus (HAV), hepatitis B virus (HBV), hepatitis C virus (HCV), herpes simplex viruses, other human herpes viruses, human rhinoviruses and other picornaviruses, influenza viruses, rhabdoviruses, and papilloma viruses.

The dual activity/cytotoxicity reporter assay system can be constructed using any transformed cell line or primary cell that can be engineered to express a reporter gene introduced by various means. The reporter gene can be introduced either stably or transiently by DNA or RNA transfection or using viral vectors. The viral-dependent and target cell-encoded reporter genes for a dual activity/cytoxicity reporter assay format can include any combination of independently identifiable reporters. Examples include the following reporter pairs: Photinus pyralis luciferase (firefly luciferase) and Renilla reniformis luciferase or derivatives thereof; firefly luciferase and secreted alkaline phosphatase; Renilla luciferase or derivatives thereof and secreted alkaline phosphatase; or beta-galacosidase and firefly luciferase; firefly luciferase and Aequora victoria green fluorescent protein (GFP) or derivatives thereof; Renilla luciferase and GFP or derivatives thereof; any combination of GFP reporters with distinct spectral emission wavelengths; and any combination of the beta-lactamase reporter with the reporters mentioned above.

B. Humanized Renilla Luciferase Nucleic Acid Molecules

Another embodiment of the invention concerns humanized Renilla luciferase genes. The Renilla luciferase (Rluc) gene is ˜30-fold less sensitive as a reporter gene in mammalian systems than firefly luciferase. One explanation for this is that the codons for Rluc are not optimal for expression in mammalian cells, as supported by comparing the codons in wild-type Rluc (SEQ ID NO:2) to those observed in highly expressed mammalian genes (Haas et al., Curr. Biol. 6:315-324 (1996)). Therefore, to increase the effective sensitivity of Rluc in mammalian cells the codons for Rluc were optimized for expression in mammalian cells.

Thus, the present invention involves a codon optimized luciferase reporter gene (HRluc) derived from the Renilla reniformis luciferase gene (Rluc) that is optimized for use in mammalian cells. This reporter gene is useful, due to its small size (˜900 nt) and sensitivity, for constructing reporter gene constructs, such as replication competent reporter viruses. Further, as described herein, this gene is useful for constructing cell lines that stably express HRluc. Such cell lines and reporter constructs are useful for the dual activity/cytotoxicity reporter assays of the present invention.

To obtain the nucleic acid molecules of the present invention and to construct recombinant vectors and cells, the present invention employs conventional microbiological and recombinant DNA techniques within the skill of the art. See, e.g., Sambrook et al. “Molecular Cloning: A Laboratory Manual” (2001); “DNA Cloning: A Practical Approach,” Volumes I and II (Glover, ed., (1985); “Current Protocols in Molecular Biology” (Ausbel et al., eds. (1993); “PCR Applications: Protocols for Functional Genomics” (Innis et al., eds. (1999); “PCR Protocols: A Guide to Methods and Applications” (Gelfand et al., eds., (1990); “Animal Cell Culture” (Freshney, ed., (1986); and Perbal, “A Practical Guide to Molecular Cloning” (1984).

As used herein, the terms “nucleic acid molecule” and “polynucleotide sequence” are intended to include DNA molecules (e.g., cDNA or genomic DNA) and RNA molecules (e.g., mRNA) and analogs of such molecules generated using nucleotide analogs. The nucleic acid molecule can be single-stranded or double-stranded.

Accordingly, the present invention provides mammalian optimized Renilla luciferase genes and methods of making and using such genes. As used herein the term “mammalian optimized Renilla luciferase gene (HRLuc)” means a gene that has been adapted for expression in mammalian cells by replacing at least one, more preferably 10, even more preferably at least 50 of the Renilla luciferase gene codons with codons that are preferred in highly expressed human genes.

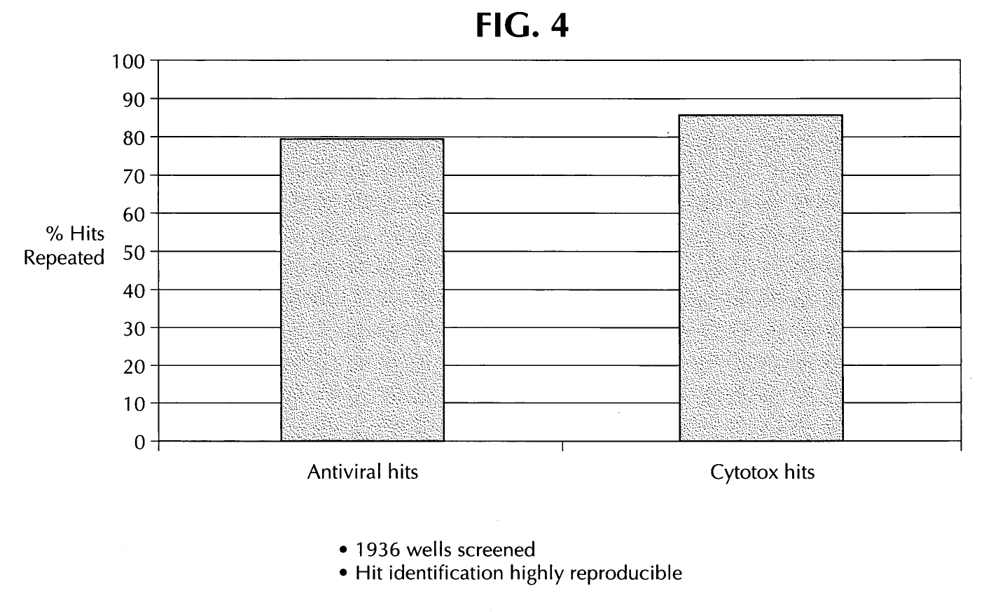

A synthetic reporter gene (HRLuc) was assembled from oligonucleotides that were amplified using PCR. The synthesized HRLuc reporter gene (FIG. 6) was introduced into a mammalian cell expression vector and its expression/activity was compared to that of the RLuc reporter in transiently transfected 293T cells. Greater than 1000-fold more luciferase activity was measured in such cells transfected with HRLuc when compared to cells transfected with Rluc (FIG. 4). These data show that HRLuc represents a reporter gene that is expressed at ˜1000-fold higher levels than Rluc in mammalian cells.

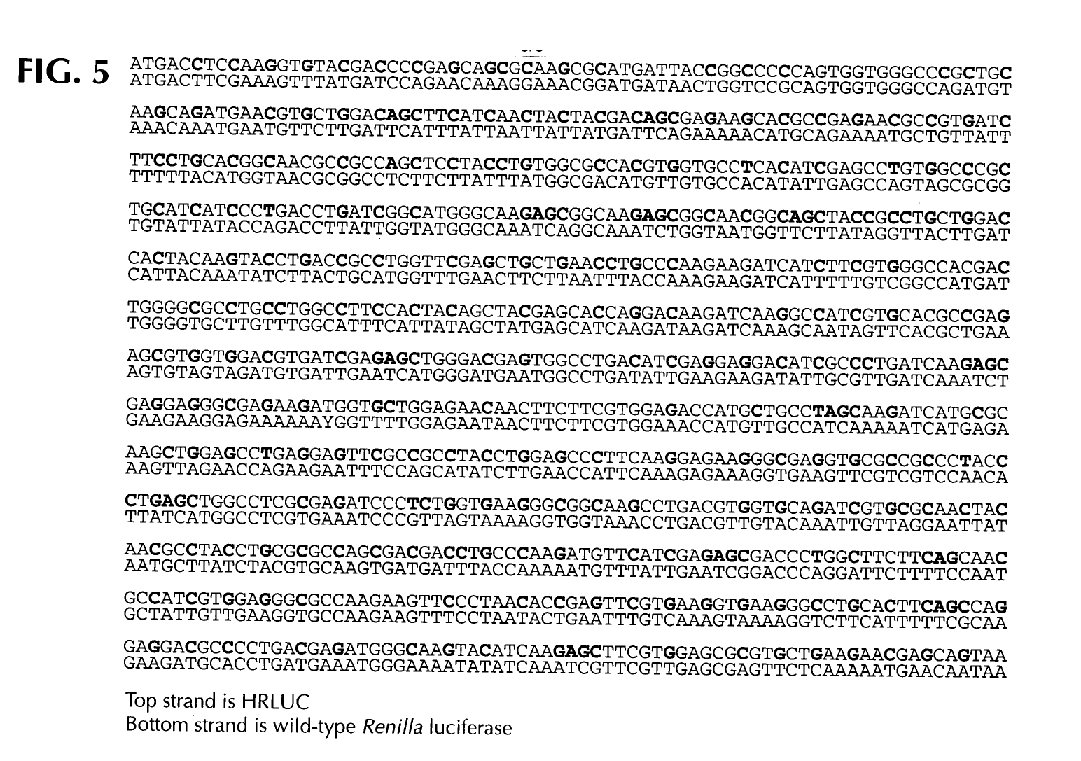

One mammalian optimized Renilla luciferase gene of the invention comprises the nucleic acid sequence of SEQ ID NO:1 (FIG. 5). Alternatively, other mammalian optimized Renilla luciferase genes may be used. For example, the artisan may refer to the codon usage information provided by Haas et al., supra, or refer to a codon usage database, such as that maintained by GenBank, in order to create a number of suitable mammalian or humanized Renilla luciferase genes.

The mammalian optimized genes of the present invention can include Renilla luciferase genes in which preferably about 10%, more preferably 15% to 30%, or even more preferably greater than 50% of the wild type luciferase codons (FIG. 6; SEQ ID NO:2) contain a humanized codon, i.e., a codon that is preferentially used in human genes.

Also contemplated in the present invention are nucleic acid molecules that are derivatives of the mammalian optimized genes of the present invention. Such derivatives include nucleic acid molecules that are less than full-length when compared to the wild-type gene and which encode functional truncations of the mammalian optimized luciferase.

The term “functional derivative” with respect to a polypeptide is a polypeptide that possesses a biological activity (either functional or structural) or an immunological characteristic that is substantially similar to a biological activity or an immunological characteristic of the humanized Renilla luciferase gene represented by SEQ ID NO:1.

Other derivatives can include nucleic acid molecules that contain mammalian optimized codons that differ from wild-type Renilla luciferase in that the nucleic acid encode a functional luciferase polypeptide with conservative amino acid changes when compared to the wild-type Renilla polypeptide. For example, the isolated replacement of a leucine with an isoleucine or valine, an aspartate with a glutamate, a threonine with a serine, or a similar replacement of an amino acid with a structurally related amino acid (i.e., a conservative replacement) will not likely have a major effect on the biological activity of the resulting molecule, especially if the replacement does not involve an amino acid at a binding site or other site of biologic activity. Standard conservative groups of amino acids are shown in parentheses using the one-letter amino acid code: nonpolar (A, V, L, I, P, M); aromatic (F, T, W); uncharged polar (G, S, T, C, N, Q); acidic (D, E); basic (K, R, H). The aromatic amino acids are sometimes considered to belong to the broader-defined nonpolar (F, W) or uncharged polar (T) groups.

In the embodiment of the present invention, HRLuc genes contain a Kozak consensus sequence upstream from the HRLuc gene sequence.

C. Vectors and Host Cells

The nucleic acid molecules of the invention are also useful for constructing recombinant vectors. The term “vector” refers to a vehicle. A preferred vector is a nucleic acid molecule that can “carry” the nucleic acid molecules of the invention. When the vector is a nucleic acid molecule, the humanized luciferase genes of the invention are covalently linked to the vector nucleic acid. Within this aspect of the invention, the term “vector” includes a plasmid, single- or double-stranded phage, a single- or double-stranded RNA or DNA viral vector, or artificial chromosome, such as a BAC, PAC, YAC, OR MAC. Vectors also include insertion vectors, used to integrate into another nucleic acid molecule sequence, such as into the cellular genome.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules of the invention. The vectors can function in prokaryotic or eukaryotic cells or in both (shuttle vectors).

Expression vectors contain cis-acting regulatory regions that are operably linked in the vector to the nucleic acid molecules of the invention such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, the host cell may supply a trans-acting factor. Furthermore, a trans-acting factor can be produced from the vector itself.

The regulatory sequences to which the nucleic acid molecules described herein can be operably linked to include promoters for directing mRNA transcription. These include, (but are not limited to), the left promoter from bacteriophage λ, the lac promoter, TRP, and TAC promoters from E. coli, the early and late promoters from SV40, the CMV immediate early promoter, the adenovirus early and late promoters, and retrovirus long-terminal repeats. However, the HRluc nucleic acid molecules of the invention can be operably linked to any promoter the artisan wishes to study or use in an assay involving a reporter construct in mammalian cells.

The term “operably linked” as used herein indicates that a gene and a regulatory sequence(s), such as a promoter, are connected in such a way as to permit gene expression when the appropriate molecules (e.g., transcriptional activator proteins or proteins which include transcriptional activation domains) are bound to the regulatory sequence(s).

In addition to control regions that promote transcription, expression vectors may also include regions that modulate transcription, such as repressor binding sites and enhancers. Examples include the SV40 enhancer, the CMV immediate early enhancer, polyoma enhancer, adenovirus enhancers, and retrovirus LTR enhancers.

In addition to containing sites for transcription initiation and control, expression vectors can also contain sequences necessary for transcription termination. Additionally, vectors may also contain signals necessary for translation such as a ribosome-binding site. Other regulatory control elements for expression include initiation and termination codons as well as polyadenylation signals. Numerous regulatory sequences that are useful in expression vectors are available in the art. Such regulatory sequences are described, for example, by Sambrook et al. (“Molecular Cloning: A Laboratory Manual.,” 3rd ed., Cold Spring Harbor Laboratory Press, Cold Spring Harbor, N.Y., (2001)).

A variety of expression vectors can be used to express a nucleic acid molecule. Such vectors include chromosomal, episomal, and virus-derived vectors, for example, vectors derived from bacterial plasmids, from bacteriophage, from yeast episomes, from yeast chromosomal elements, including yeast artificial chromosomes, from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., supra.

The regulatory sequence may provide constitutive expression in one or more host cells (i.e. tissue specific) or may provide for inducible expression in one or more cell types such as by temperature, nutrient additive, or exogenous factor such as a hormone or other ligand. Selection of suitable vectors providing for constitutive and inducible expression in prokaryotic and eukaryotic hosts is within the purview of those of ordinary skill in the art.

The nucleic acid molecules can be inserted into the vector nucleic acid by known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are known to those of ordinary skill in the art.

The vector containing the appropriate nucleic acid molecule can be introduced into an appropriate host cell for propagation or expression using known techniques. Host cells can include bacterial cells including, but not limited to, E. coli, Streptomyces, and Salmonella typhimurium, eukaryotic cells including, but not limited to, yeast, insect cells, such as Drosophila, animal cells, such as COS, HEK 293, MT-2T, CEM-SS, and CHO cells, and plant cells.

In an embodiment of the invention, the nucleic acid molecules described herein are expressed in mammalian cells using mammalian vector systems including those described herein. For example, the luciferase genes of the invention can be operably linked to a promoter expressed or potentially expressed in mammalian cells. Because of the high sensitivity of the luciferase genes of the invention, the promoter can be a weak promoter. The promoter need not be from a mammalian source and can include promoters from microorganisms, such as viruses or bacteria.

The luciferase genes of the invention can be used to construct a reporter virus. The reporter virus can be derived from any virus for which an infectious and/or replication competent molecular clone is available. By “infectious and/or replication competent molecular clone” is meant any cDNA containing viral sequences that encode the potential to direct replication of either full-length or subgenomic viral cDNAs via viral promoters and/or replication signal sequences. In addition, the luciferase genes of the invention can be used to construct a reporter virus derived from any virus generated through homologous recombination events in an infected cell. Thus, the luciferase genes of the invention are suitable for constructing a reporter virus in human immunodeficiency viruses (HIV-1 and HIV-2), hepatitis A virus (HAV), hepatitis B virus (HBV), hepatitis C virus (HCV), herpes simplex viruses, other human herpes viruses, human rhinoviruses and other picornaviruses, influenza viruses, rhabdoviruses, and papilloma viruses.

The humanized luciferase genes can also be used to construct reporter systems in other pathogenic organisms known to infect mammalian cells, e.g., bacteria, such as E. coli, Salmonella, Yersinia, Listeria, Staphylococcus aerus, Shigella, and Chlamydia, fungi, and protozoa, such as Leishmania.

The invention also relates to recombinant host cells containing the vectors described herein.

The term “recombinant host cell” refers to a cell that has been altered to contain a new combination of genes or nucleic acid molecules.

The recombinant host cells are prepared by introducing the vector constructs described herein into the cells by techniques readily available in the art. These include calcium phosphate transfection, DEAE-dextran-mediated transfection, cationic lipid-mediated transfection, electroporation, transduction, infection, lipofection, and other techniques, such as those found in Sambrook et al. supra.

Host cells can contain more than one vector. Thus, different nucleotide sequences can be introduced on different vectors to the same cell. Similarly, the nucleic acid molecules can be introduced either alone or with other nucleic acid molecules that are not related to the nucleic acid molecules, such as those providing trans-acting factors for expression vectors. When more than one vector is introduced into a cell, the vectors can be introduced independently, co-introduced or joined to the polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

The recombinant host cells containing the nucleic acid molecules of the invention have a variety of uses. For example, the cells are useful for studying gene regulation and for screening assays for drug discovery. Preferred cells for expression purposes will be mammalian cells. Other cell types, such as bacterial, yeast, fungal, insect, nematode, and plant cells, are also possible. Examples of suitable mammalian recombinant host cells include VERO, HeLa, CHO, COS, BHK, HepG2, 3T3, or other mammalian cell lines.

In yet another embodiment, the luciferase genes of the invention are integrated into the genome of a mammalian cell or host. Exemplary integration events that are random or integration events that are site-specific, such as through use of CRE-mediated site-specific recombination. Once integrated into the genome, the luciferase gene can be constitutively expressed or expressed through the use of an inducible promoter. Such cells are useful for the dual activity/cytotoxicity reporter assays of the invention, for example, as a reporter to monitor cell viability.

The luciferase genes of the invention can also be used to construct fusion proteins. The construction of fusion proteins is known in the art, e.g., see Day et al., Biotechniques 25:848-50, 852-4, 856 (1998); Kobatake et al., Anal Biochem. 208:300-305 (1993)); and Wang et al., Mol. Gen. Genet. 264:578-87 (2001)).

The present invention is also drawn to kits. Such kits can comprise a humanized luciferase gene, for example, carried in a vector. The kit may also contain instructions for use of the humanized luciferase gene.

### Exemplary Methods and Materials

Preparation of Cells:

HeLa cells, CEM-SS cell, MT-2 cells, and HEK 293 cells (obtained through the National Institutes of Health AIDS Research and Reference Reagent Program, Bethesda, Md.) were propagated in Dulbecco's Modified Eagle Medium (DMEM; Life Technologies, Gaithersburg, Md.) containing 10% fetal bovine serum (FBS; HyClone, Logan, Utah). HeLa HRLuc cells (see below) were propagated in DMEM (Life Technologies) containing 10% FBS (HyClone) and 1 mg/ml Geneticin (Life Technologies).

Virus:

The HIV-1 NL4-3 proviral clone and HIV-1 RF virus strain were obtained through the National Institutes of Health AIDS Research and Reference Reagent Program.

Preparation of Compounds:

Capravirine (CPV) was synthesized at Shionogi & Co., Ltd. (Osaka, Japan) and at Agouron Pharmaceuticals, Inc. (San Diego, Calif.). Nelfinavir (NFV), L-798906, and L-731988 were synthesized at Agouron Pharmaceuticals, Inc. (San Diego, Calif.). Nevirapine (NVP), delavirdine (DLV), efavirenz (EFV), lamivudine (3TC), stavudine (d4T), and saquinavir (SQV) were provided by Boehringer Ingelheim Pharmaceuticals (Ridgefield, Conn.), Pharmacia and Upjohn (Kalamazoo, Mich.), DuPont Merck Pharmaceutical Company (Wilmington, Del.), Glaxo Wellcome (Research Triangle Park, N.C.), Bristol-Myers Squibb (Wallingford, Conn.), and Roche Laboratories (Welwyn, UK), respectively. 3′-Azido-3′-deoxythymidine (AZT) and didanosine (ddI) were purchased from Sigma-Aldrich (St Louis, Mo.).

High-Throughput Assays:

Test compound was added to HeLa HRLuc target cells, seeded in 96-well plates at a cell density of 1×10⁴ cells per well in DMEM (Life Technologies) containing 10% FBS (HyClone), at a final compound concentration of 10 μM and final dimethyl sulfoxide (DMSO; Sigma-Aldrich, St. Louis, Mo.) concentration of 1%. For the mock screen assays, either test compound (added to 2-4 wells per plate) or DMSO alone was added to HeLa HRLuc target cells in 96-well plates (1×10⁴ cells per well). Compound or DMSO-treated HeLa HRLuc target cells were then infected with VSV/HIVLuc at an moi of 0.03. Seventy-two hours after infection, the activities of firefly luciferase (the viral-encoded reporter) and Renilla luciferase (the target cell-encoded reporter) were measured in the infected cells using the Dual-Luciferase Reporter Assay System according to the manufacturer's protocol (Promega). Data from the reporter gene measurements were expressed as the percent inhibition of reporter gene activity in infected compound-treated cells relative to that of infected compound-free cells. In addition, the Z′ coefficient was calculated from the no drug control wells using the equation 1−[(3×SD positive control)−(3×SD negative control)/(mean positive control−mean negative control)] (Zhang et al., 1999, supra).

XTT Cytotoxicity Assay:

Test compounds were added to HeLa HRLuc target cells in 96 well plates (1×10⁴ cells per well) at a final compound concentration of 10 μM and final DMSO (dimethyl sulfoxide) concentration of 1%. Seventy-two hours later, 50 μl of XTT (1 mg/ml XTT tetrazolium, 0.02 nM phenazine methosulfate) was added to the wells and the plate was reincubated for four hours (Weislow et al., 1989, supra). Viability, as determined by the amount of XTT formazan produced, was quantified spectrophotometrically by absorbance at 450 nm. Data from XTT cytotoxicity assays were expressed as the percent of formazan produced in compound-treated cells compared to formazan produced in wells of uninfected, compound-free cells.

PCR Methods:

Fifty μl polymerase chain reaction (PCR) mixtures were prepared containing 100 ng of the appropriate oligonucleotide primers, 250 ng of template DNA, a final concentration of 200 nM for each dNTP (Roche, Indianapolis, Ind.), 2.5 units of Herculase Blend polymerase (Stratagene, San Diego, Calif.), and a 1:10 dilution of the 10× Herculase Blend reaction buffer provided by the manufacturer (Stratagene). PCR reactions were initiated by incubation at 95° C. for 2 minutes. PCR amplification was then carried out for 30 iterative cycles with each cycle consisting of the following steps: (1) 30 seconds at 95° C., (2) 30 seconds at 50° C., and (3) 1 minute at 60° C.

Dual-Luciferase Reporter Assay:

In Promega's Dual-Luciferase® Reporter (DLR) Assay System, the activities of firefly and Renilla luciferases are measured sequentially from a single sample. The firefly luciferase reporter is measured first by adding Luciferase Assay Reagent II (LAR II) to generate a luminescent signal. LAR II is a proprietary solution supplied with the DLR Assay Kit. After measurement of this signal, the reaction is stopped; and, the Renilla luciferase reaction is initiated by simultaneously adding Stop & Glo® Reagent (a proprietary solution supplied with the Promega kit) to the same tube.

In general, the DLR Assay is performed as follows. The growth medium is removed from the cultured cells. Into each culture container is added 1× Passive Lysis Buffer (PLB), which is a proprietary solution obtained with the DLR Assay Kit. For passive lysis the culture plates are gently rocked or shaken for 15 minutes at 25° C.

To measure firefly luciferase activity, 100 μl of LAR II is added to each well of the microtiter plate. The microtiter plate is placed in a luminometer for measurement of luciferase activity. After measurement of the firefly luciferase activity, Renilla luciferase activity is measured after adding 100 μl of Stop & Glo® Reagent to the microtiterplate. The sample is placed into a luminometer to measure Renilla luciferase activity.

Transfections Using LIPOFECTAMINE Plus Reagent (Life Technologies):

The day before transfection the cells are trypsinized and counted. The cells are then plated so that they are 70%-90% confluent on the day of transfection. The nucleic acid molecule to be transfected is diluted into Dulbecco's Modified Eagle Medium (DMEM) (or other appropriate medium) without serum. The PLUS Reagent (Life Technologies) is mixed and added to the diluted nucleic acid molecule and mixed. This solution is incubated at room temperature for 15 min. In another tube, the LIPOFECTAMINE Reagent is diluted in DMEM (or other appropriate medium) without serum and mixed. The nucleic acid molecule-Plus solution is combined with the diluted LIPOFECTAMINE Reagent, mixed and incubated for 15 min. at room temperature. While the LIPOFECTAMINE Reagent-nucleic acid molecule-PLUS complexes are forming, the medium on the cells is replaced with transfection medium. The LIPOFECTAMINE Reagent-nucleic acid molecule-PLUS complexes are added to the cells containing fresh medium. The complexes are gently mixed into the medium and incubated at 37° C. with 5% CO₂ for 3 h. After this incubation, the volume of medium on the cells is increased to a normal volume. If the transfection was without serum, serum is added to bring the final concentration to that of normal growth medium. The cell medium can be replaced with fresh complete medium to facilitate cell growth. Cells are assayed or stained in situ for reporter gene activity 24 h to 48 h after transfection, depending on cell type and promoter activity. For stable expression, the cells can be passaged into fresh culture medium 1 day after the start of transfection and at 2 days the selection antibiotic can be added.

The following examples are given for the purpose of illustrating various embodiments and features of the invention.

### EXAMPLE 1

### Construction of a Humanized Renilla Luciferase Gene

HRLuc construction: The HRLuc reporter gene, which was derived from the Renilla reniformis luciferase reporter gene and codon optimized for high level expression in mammalian cells, was constructed by polymerase chain reaction (PCR) using synthetic oligonucleotide primers and templates. Template 1 (5′-ATG ACC TCC AAG GTG TAC GAC CCC GAG CAG CGC AAG CGC ATG ATT ACC GGC CCC CAG TGG TGG GCC CGC TGC AAG-3′) (SEQ ID NO:3) was amplified with Primers HRLA (5′-GAA TCA TCT AGA ATG ACC TCC AAG GTG TAC GAC CCC GA-3′) (SEQ ID NO:4) and HRLB (5′-GTT CAT GAA TTC CTT GCA GCG GGC CCA CCA CTG-3′) (SEQ ID NO:5), digested with the restriction endonulceases XbaI and EcoRI and ligated to pGEM 3fz+ (Promega) digested with the same enzymes to form pHRLI.

Templates 2 (5′-GTG CTG GAC AGC TTC ATC AAC TAC TAC GAC AGC GAG AAG CAC GCC GAG AAC GCC GTG ATC TTC CTG CAC GGC AAC GCC GCC AGC TCC TAC CTG TGG CGC C-3′) (SEQ ID NO:6) and 3 (5′-CGC TCT TGC CCA TGC CGA TCA GGT CAG GGA TGA TGC AGC GGG CCA CAG GCT CGA TGT GAG GCA CCA CGT GGC GCC ACA GGT AGG AGC TGG CGG CGT TGC C-3′) (SEQ ID NO:7) were annealed and amplified with primers HRLC (5′-GAA TCA TCT AGA TGG GCC CGC TGC AAG CAG ATG AAC GTG CTG GAC AGC TTC ATC AAC TAC TAC GA-3′) (SEQ ID NO:8) and HRLD (5′-ACT TGT AGT GGT CCA GCA GGC GGT AGC TGC CGT TGC CGC TCT TGC CGC TCT TGC CCA TGC CGA TCA GGT C-3′) (SEQ ID NO:9) to generate amplified product HRLII, while templates 4 (5′-CGA GCT GCT GAA CCT GCC CAA GAA GAT CAT CTT CGT GGG CCA CGA CTG GGG CGC CTG CCT GGC CTT CCA CTA CAG CTA CGA GCA CCA GGA CAA GAT CA-3′) (SEQ ID NO:10) and 5 (5′-CCT CCT CGA TGT CAG GCC ACT CGT CCC AGC TCT CGA TCA CGT CCA CCA CGC TCT CGG CGT GCA CGA TGG CCT TGA TCT TGT CCT GGT GCT CGT AGC TGT-3′) (SEQ ID NO:11) were amplified with primers HRLE (5′-ACC GCC TGC TGG ACC ACT ACA AGT ACC TGA CCG CCT GGT TCG AGC TGC TGA ACC TGC CCA AGA AGA TCA T-3′) (SEQ ID NO:12) and HRL F (5′-CAT GAT GM TTC TGA TCA GGG CGA TGT CCT CCT CGA TGT CAG GCC ACT CGT C-3′) (SEQ ID NO:13) to generate amplified product HRLIII. The HRLII and HRLIII amplified products were introduced into a recombinant PCR reaction and amplified using primers HRLC and HRLF. The resulting product HRLII/III was digested with the restriction endonulceases XbaI and EcoRI and ligated to pGEM 3fz+ digested with the same enzymes to form pHRLII/III.

Templates 6 (5′-GAG AAG ATG GTG CTG GAG AAC AAC TTC TTC GTG GAG ACC ATG CTG CCT AGC AAG ATC ATG CGC AAG CTG GAG CCT GAG GAG TTC GCC GCC TAC CTG G-3′) (SEQ ID NO:14) and 7 (5′-CTT CAC CAG AGG GAT CTC GCG AGG CCA GCT CAG GGT AGG GCG GCG CAC CTC GCC CTT CTC CTT GAA GGG CTC CAG GTA GGC GGC GAA CTC CTC AGG CTC-3′) (SEQ ID NO:15) were annealed and amplified with primers HRLG (5′-GAT ACA TCT AGA TGA TCA AGA GCG AGG AGG GCG AGA AGA TGG TGC TGG AGA ACA ACT TCT TC-3′) (SEQ ID NO:16) and HRLH (5′-AGT TGC GCA CGA TCT GCA CCA CGT CAG GCT TGC CGC CCT TCA CCA GAG GGA TCT CGC GAG GCC AGC T-3′) (SEQ ID NO:17) to generate amplified product HRLIV, while templates 8 (5′-CGC GCC AGC GAC GAC CTG CCC AAG ATG TTC ATC GAG AGC GAC CCT GGC TTC TTC AGC AAC GCC ATC GTG GAG GGC GCC AAG AAG TTC CCT AAC ACC GAG T-3′) (SEQ ID NO:18) and 9 (5′-AGC TCT TGA TGT ACT TGC CCA TCT CGT CAG GGG CGT CCT CCT GGC TGA AGT GCA GGC CCT TCA CCT TCA CGA ACT CGG TGT TAG GGA ACT TCT TGG CGC C-3′) (SEQ ID NO:19) were amplified with primers HRLI (5′-CCT GAC GTG GTG CAG ATC GTG CGC AAC TAC AAC GCC TAC CTG CGC GCC AGC GAC GAC CTG CCC MG ATG-3′) (SEQ ID NO:20) and HRL J (5′-TCG ATA GAA TTC TTA CTG CTC GTT CTT CAG CAC GCG CTC CAC GAA GCT CTT GAT GTA CTT GCC CAT CTC GTC-3′) (SEQ ID NO:21) to generate amplified product HRLV. The HRLIV and HRLV amplified products were introduced into a recombinant PCR reaction and amplified using primers HRLG and HRLJ. The resulting product HRLIV/V was digested with the restriction endonulceases XbaI and EcoRI and ligated to pGEM 3fz+ digested with the same enzymes to form pHRLIV/V.

Each of the HRLuc fragments contained in pHRLucI, pHRLucII/III and pHRLucIV/V was subjected to sequence analysis to confirm sequence identity. Following sequence confirmation, pHRLIV/V was digested with the restriction enzymes BclI and EcoRI and the resulting 443 bp containing the HRLuc sequences 493 to 936 was ligated to pHRLII/III digested with the same restriction enzymes to generate pHRLII/V. pHRLII/V was then digested with the restriction enzymes ApaI and EcoRI and the resulting 870 bp fragment containing HRLuc sequences 66 to 936 was ligated to pHRLI digested with the same enzymes to generate pHRLuc.

### EXAMPLE 2

### Comparison of HRLuc Activity to Rluc Activity

To test expression of the constructed HRLuc gene in mammalian cells, pCMVHRLuc was constructed by digesting pHRLuc with XbaI and EcoRI and introducing the resulting 936 bp HRLuc gene into pcDNA3.1+(Invitrogen, Carlsbad, Calif.) digested with the same enzymes.

To determine whether the constructed HRLuc gene directs higher levels of Renilla luciferase expression, the reporter gene activity in cells transfected with an HRLuc expression vector (pCMVHRLuc) was compared to that observed in cells transfected with a similar expression vector encoding the non-optimized RLuc gene (pCMVNRLuc). HEK 293 cells were co-transfected with either pCMVHRLuc or pCMVNRLuc and a transfection control vector encoding the firefly luciferase reporter gene. Approximately 72 hours after transfection, cells were harvested, lysed, and a portion of the cell lysates was analyzed for Renilla luciferase and firefly luciferase activity, using the Promega dual reporter system. The Renilla luciferase activity derived from HRLuc or NRLuc was expressed as log relative light units and normalized for transfection efficiency using the firefly luciferase activity measured in the respective cell lysates. As shown in FIG. 4, about a 1000-fold increase in luciferase activity was measured in the cells transfected with the HRLuc vector when compared to that observed in cells transfected with the NRLuc expression vector. These data demonstrate that the HRLuc gene directs ˜1000-fold higher levels of luciferase expression when compared to the non-optimized RLuc gene (NRLuc).

### EXAMPLE 3

### Construction of HRLuc Target Cell Lines

HeLa target cells were constructed that constitutively express a Renilla luciferase gene codon optimized for high-level expression in mammalian cells (HRLuc). Sequences corresponding to the HRLuc reporter gene (SEQ ID NO:1) were removed from pHRLuc using the Xba I and Xho I restriction endonucleases (New England BioLabs, Beverly, Mass.) and ligated to the pcDNA 3.1 (Life Technologies) expression vector digested with the Nhe I and Xho I restriction endonucleases (New England BioLabs). The resulting construct pcDNA/HRLuc encodes the HRLuc reporter gene under the control of the CMV immediate early promoter as well as the neomycin resistant gene under the control of the SV-40 promoter. HeLa cells were transfected with pcDNA3.1/HRLuc using LIPOFECTAMINE Plus according to the manufacturer's protocol (Life Technologies). Three days after transfection, selection was initiated by adding G418 (Geneticin; Life Technologies) to the tissue culture media at a final concentration of 1 mg/mL. Two weeks after growth in selection, G418 resistant cells were harvested, and cell clones were isolated by limiting dilution. Individual clones were propagated and tested for Renilla luciferase expression using the Dual-Luciferase® Reporter Assay System according to the manufacturer's protocol (Promega). HeLa HRLuc clones that exhibited high levels of Renilla luciferase activity were characterized further and a single HeLa HRLuc clone was selected for use in the experiments described herein.

### EXAMPLE 4

### Construction of HIV-1 Reporter Virus (VSV/HIVLuc)

An HIV-1 single-cycle infectious reporter virus was constructed based on a design previously published (Akkina et al., J. Virol. 70(4):2581-2585 (1996)). A two-nucleotide insertion was introduced in the envelope coding region of pNL4-3 (nucleotide position 6401) using polymerase chain reaction (PCR)-based mutagenesis [PCR-based mutagenesis was carried out using the QuickChange™ site-directed mutagenesis kit (Stratagene, La Jolla, Calif.) according to the manufacturer's protocol], which resulted in a frame shift mutation in envelope sequences of pNL4-3 (pNL4-3_Env). The primers used for the mutagenesis were as follows: (1) 5′-CCT CTG TAT CAT ATA TGC TTT A-3′ (SEQ ID. NO:22) and (2) 5′-TAA AGC ATA TAT GAT ACA GAG G-3′ (SEQ ID NO:23). The SV-40 origin of DNA replication was then introduced into pNL4-3_Env at the unique Nco I restriction enzyme site (pNL4-3 nucleotide position 10568). The firefly luciferase gene was subsequently inserted in place of Nef coding sequences between the unique Xho I site in pNL4-3 (nucleotide position 8887) and an Mlu I site introduced by site directed mutagenesis at nucleotide position 9008, resulting in the construction of pNLuc_env. To generate single-cycle infectious virus, pNLuc_env was co-transfected with a vesicular stomatitis virus (VSV) envelope expression vector (obtained from Stratagene) into HEK 293 cells using LIPOFECTAMINE Plus according to the manufacturer's protocol (Life Technologies). Forty-eight to seventy-two hours after transfection, single-cycle infectious HIV reporter virus was harvested from the supernatants of transfected cells. The supernatants of transfected cells were harvested, clarified by centrifugation (500×g), and filtered through a 0.45-micron membrane (Corning, Acton, Mass.). Titers (TCID₅₀) of the resulting viral stocks were determined after infecting HeLa HRLuc target cell lines with serial dilutions of the viral stocks (Johnson and Byrington, 1990, supra) and measuring firefly luciferase activity 72 hours after infection using a firefly reporter gene assay kit (Promega, Madison, Wis.).

### EXAMPLE 5

### Antiviral Activity and Cell Cytotoxicity of HIV-1 Inhibitors Measured Using a Dual Activity/Cytotoxicity Reporter Assay

The VSV/HIVLuc single-cycle infectious reporter virus described in Example 5 and the HeLa cytotoxicity control cell line described in Example 4 were used to analyze specific antiviral activities and cytotoxicities of known HIV-1 inhibitors. Expression of the firefly luciferase and Renilla luciferase reporters served as an indicator of viral infection and compound-mediated cytotoxicity, respectively.

To evaluate the antiviral activities and cytotoxicities of HIV-1 RT and IN inhibitors in the dual activity/cytoxicity reporter assay system, half-log dilutions of test compounds were added to HeLa HRLuc target cells, seeded in 96-well plates at a cell density of 1×10⁴ cells per well in DMEM (Life Technologies) containing 10% FBS (HyClone). Compound-treated or compound-free HeLa HRLuc target cells were then infected with VSV/HIVLuc at an moi of 0.03. In the case of nucleoside reverse transcriptase inhibitors (AZT, 3TC, d4T, and ddI), the compound-treated cells were incubated at 37° C. for one hour prior to virus addition. Seventy-two hours after infection, the activities of firefly luciferase (the viral-encoded reporter) and Renilla luciferase (the target cell-encoded reporter) were measured in the infected cells using a dual-luciferase reporter assay system according to the manufacturer's protocol (Promega).

Data from the reporter gene measurements were expressed as the percent of reporter gene activity in infected compound-treated cells relative to that of infected, compound-free cells. The fifty percent effective concentration (EC₅₀) was calculated as the concentration of compound that caused a decrease in the percentage of the virally encoded reporter gene activity in infected, compound-treated cells to 50% of that produced in infected, compound-free cells. The 50% cytotoxicity concentration (CC₅₀) was calculated as the concentration of compound that effected a decrease in the percentage of the target cell-encoded reporter gene activity in infected, compound-treated cells to 50% of that produced in infected, compound-free cells. The therapeutic index (TI), which is a measurement of the specific antiviral activity of a compound, was calculated by dividing the cytotoxicity (CC₅₀) by the antiviral activity (EC₅₀). As shown in Table 1, the antiviral activities (EC₅₀) and cytotoxicities (CC₅₀) of the NNRTIs (CPV, DLV, EFV, and NVP), NRTIs (nucleoside reverse transcriptase inhibitors) (AZT, DDI, 3TC, and D4T) and IN inhibitors (L-731988 and L-798906) tested in the dual activity/cytotoxicity assay were similar to those observed in cell-protection assays, using HIV-1 RF and CEM-SS cells (see below).

The antiviral activities of compounds were also evaluated in cell protection assays by the XTT dye reduction method. CEM-SS subject cells were infected with HIV-1 RF virus at an moi of 0.025 to 0.819 or mock infected with medium only and added at 2×10⁴ cells per well into 96-well plates containing half-log dilutions of test compounds. Six days later, 50 μl of XTT (1 mg/ml XTT tetrazolium, 0.02 nM phenazine methosulfate) was added to the wells and the plate was reincubated for four hours. Viability, as determined by the amount of XTT formazan produced, was quantified spectrophotometrically by absorbance at 450 nm.

Data from cell protection assays were expressed as the percent of formazan produced in compound-treated cells compared to formazan produced in wells of uninfected, compound-free cells. The fifty percent effective concentration (EC₅₀) was calculated as the concentration of compound that caussed an increase in the percentage of formazan production in infected, compound-treated cells to 50% of that produced by uninfected, compound-free cells. The 50% cytotoxicity concentration (CC₅₀) was calculated as the concentration of compound that decreased the percentage of formazan produced in uninfected, compound-treated cells to 50% of that produced in uninfected, compound-free cells (Table 1).

### EXAMPLE 6

### Dual Activity/Cytotoxicity Reporter Assay Used in a High-Throughput Screening Format

HIV-1 Mock Screen

In addition to determining the antiviral activities and cytotoxicities of known inhibitors, the dual activity/cytotoxicity reporter assay can be used to identify potential drugs in a high-throughput format. As an initial demonstration of assay suitability for high-throughput screening, coefficients of variation and screening window coefficients (Z′ value) were calculated for each of the assay endpoints. The Z′ coefficient was calculated from the no drug control wells using the equation 1−[(3×SD positive control)−(3×SD negative control)/(mean positive control-mean negative control)] (Zhang et al., 1999, supra). As seen in FIG. 1, HeLa HRLuc target cells were infected with VSV/HIVLuc in the presence or absence of a compound. Seventy-two hours after infection, the activities of firefly luciferase (the viral-encoded reporter) and Renilla luciferase (the target cell-encoded reporter) were measured in the infected cells using a Dual-Luciferase® reporter assay. The signal to background of the viral reporter was ˜10³ relative light units (RLUs), while the signal to background of the cytotoxicity control reporter is ˜10⁴ RLUs. The Z′ value is reflective of the dynamic range as well as the variation of the assay and is a useful tool for assay comparisons and assay quality determinations (Zhang et al., 1999, supra). Typically a Z′ value >0.5 is considered favorable for high-throughput screening. The antiviral and cytotoxicity components of the assay exhibited coefficients of variation (CV) of 19.5% and 13.4%, respectively, and Z′ values of 0.72 and 0.88, respectively (FIG. 1). Therefore, the low CVs and favorable Z′ values suggest that the dual activity/cytotoxicity reporter assay is suitable for high-throughput screening. The expected profiles of reporter gene expression for antiviral agents, cytotoxic agents, and agents that exhibit no activity are also shown in FIG. 1.

To further demonstrate that the dual activity/cytotoxicity reporter assay is suitable for high-throughput screening, 96-well mock screening plates were evaluated using the VSV/HIVLuc reporter virus and the HeLa HRLuc cytotoxicity control cell line. The mock plates were designed to mimic an actual compound library screen and thus, contained DMSO in each well at a final concentration equivalent to that encountered in a typical cell-based screen (1% final). In addition, the mock plates contained known HIV-1 inhibitors or cytotoxic compounds distributed randomly in 1-2 wells per plate. For the experiment shown in FIG. 2, each of the three compounds was introduced into five wells distributed randomly across five different plates, resulting in a total of 10 mock plates. Compound 1 was introduced at the predicted antiviral 50% effective concentration (EC₅₀) for that compound. Compound 2, which exhibited both antiviral activity and subtle cytotoxicity, was introduced at a concentration corresponding to its predicted antiviral 60% effective concentration and 25% cytotoxic concentration, while compound 3 was introduced at the predicted 80% cytotoxic concentration. Seventy-two hours after infection, the activities of firefly luciferase (the viral-encoded reporter) and Renilla luciferase (the target cell-encoded reporter) were measured in the infected cells using the dual reporter assay. Data representing the reporter gene measurements from such mock screening plates were expressed as the percent inhibition of reporter gene activity in infected compound-treated wells relative to that of infected compound-free wells. The mean percent inhibition values and corresponding standard deviations were then calculated for the antiviral and cytotoxicity measurements for each compound and plotted (FIG. 2). The data show a highly reproducible measurement of the antiviral activities and/or cytotoxicities of the test compounds across microtiter plates (interplate standard deviation <14%). In addition, the levels of reporter gene inhibition observed for the antiviral and cytotoxicity components of the assay were similar to that expected for all three compounds. Therefore, these data demonstrate the potential for reproducible identification of specific antiviral compounds using the dual activity/cytoxicity reporter assay in a high-throughput format.

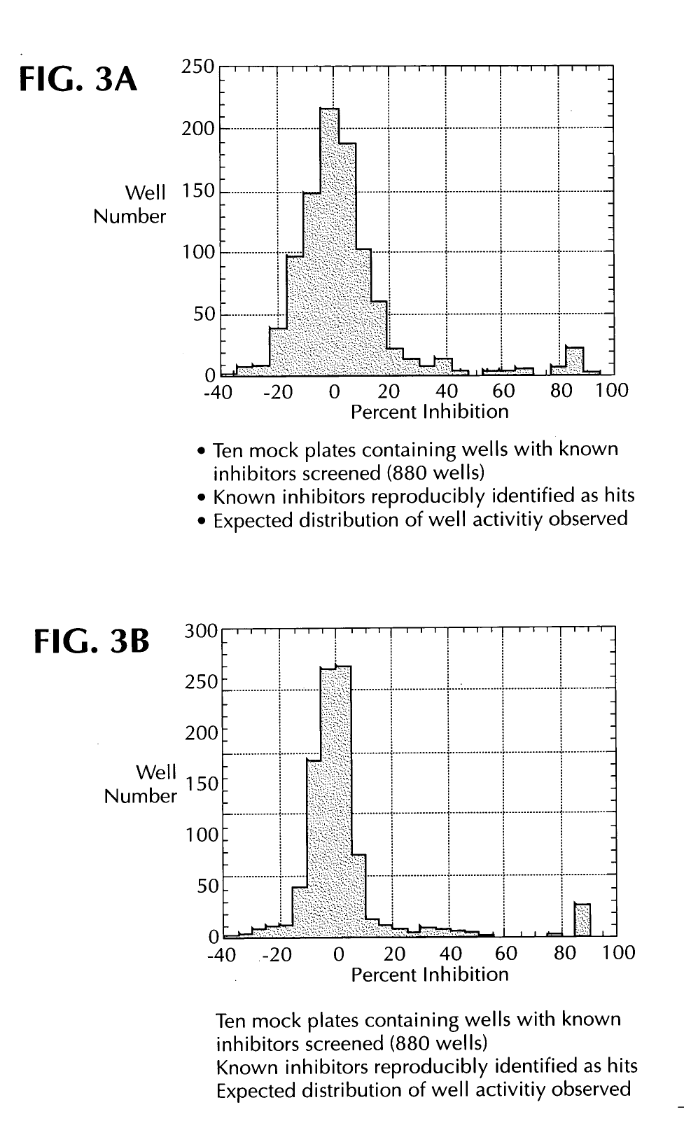

As a separate demonstration of assay reproducibility and to demonstrate that inhibitor identification was specific for wells containing compound in the 10-plate mock screen, the activity observed in each well (880 wells) was analyzed further. Either test compound (added to 2-4 wells per plate) or DMSO alone was added to HeLa HRLuc target cells in 96-well plates. Compound or DMSO-treated HeLa HRLuc target cells were infected with VSV/HIVLuc, and the activities of firefly luciferase (the viral-encoded reporter) and Renilla luciferase (the target cell-encoded reporter) were measured in the infected cells using a Dual-Luciferase® reporter assay seventy-two hours after infection. Percent inhibition values relative to the no compound control were calculated for the antiviral and cytotoxicity measurements in each well and plotted in a histogram format. As shown in FIG. 3, which represents the distribution of well activity in a mock screen, the majority of wells exhibited percent inhibition values between −20 and 20% for the antiviral measurements and between −15 and 15% for the cytotoxicity measurements. These data closely agree with the coefficient of variation (CV) values calculated for each of the assay endpoints (19.5% and 13.4% for the antiviral and cytotoxicity measurements, respectively). The majority of outlier values represent wells that contain compounds with known activities. These data emphasize the reproducibility of both endpoints of the assay and show inhibitory activities identified in the mock screen as significant and compound specific.

### EXAMPLE 7

### Compound Library Screening Using a Dual Activity/Cytotoxicity Reporter Assay

The utility of the assay system for high-throughput screening of a random compound library can be evaluated using the dual activity/cytotoxicity assay described under Examples 7 and 8. In such experiments, the reproducibility of inhibitor identification for both the antiviral and cytotoxicity measurements can be determined in two experiments. For the purposes of this analysis, an antiviral inhibitor or cytotoxic compound can be defined as any compound that effects a reduction in the activity of either the viral-encoded or target cell-encoded reporter gene, respectively, to levels that are ≦40% of that observed for the no compound control wells (i.e., ≧60% inhibition). Inhibitors identified after screening the random compound library can be retested in a similar screen to determine the reproducibility of inhibitor identification. Similarly, compounds identified as cytotoxic can be retested to determine the reproducibility of cytotoxic identification.

To demonstrate a correlation between inhibition of target cell reporter gene activity and cytotoxicity, the compounds screened using the dual activity/cytotoxicity reporter assay system can also be analyzed for effects on cell viability using an XTT dye reduction method (Weislow et al., 1989, supra). Test compounds can be added to HeLa HRLuc target cells at a final compound concentration of 10 μM and final DMSO concentration of 1%. Seventy-two hours later, cell viability can be determined using the XTT dye reduction method. Results can be calculated as percent viable cells in compound-treated wells relative to that observed in compound-free wells. A comparison can then be made between the cytoxicity results of the dual activity/cytotoxicity reporter assay as compared to the cytotoxicity results determined using XTT analysis.

To demonstrate the utility of the assay system for distinguishing specific antiviral inhibitors from non-specific inhibitors or cytotoxic compounds in a high-throughput format, microtiter plates containing compounds generated by combinatorial chemistry can be screened for antiviral activity and cytotoxicity at a final concentration of 10 μM using the dual activity/cytotoxicity reporter assay as described under Examples 7 and 8. For purposes of this assay, an inhibitor or cytotoxic compound can be defined as any compound that effects a reduction in the activity of either the viral-encoded or target cell-encoded reporter gene to levels that are ≦40% of that observed for the no compound control wells (i.e., ≧60% inhibition).

The foregoing description has been provided to illustrate the invention and its preferred embodiments. The invention is intended not to be limited by the foregoing description, but to be defined by the appended claims.

1. A method for evaluating both activity and cytotoxicity of a compound in the same population of cells comprising the steps of:
(a) providing a target cell comprising a first reporter gene that monitors cell viability;
(b) adding a test compound to said population of cells;
(c) introducing a second reporter gene into said cells, wherein said second reporter gene is different than said first reporter gene, and said second reporter gene is indicative of the activity of a microorganism or cell-receptor and monitors the activity of said test compound;
(d) measuring the activity of said first reporter gene and said second reporter gene; and
(e) comparing the activity of said first reporter gene and said second reporter gene measured in step (d) to the activity of said first reporter gene and said second reporter gene measured in the absence of said test compound.

2. A method for evaluating activity and cytotoxicity of a compound in the same population of cells comprising the steps of:
(a) providing a target cell comprising a first reporter gene that monitors cells viability and a second reporter gene that is indicative of the activity of a microorganism or cell-receptor, wherein said second reporter gene is different than said first reporter gene;
(b) adding a test compound to said cells;
(c) measuring the activity of said first reporter gene and said second reporter gene; and
(d) comparing the activity of said first reporter gene and said second reporter gene measured in step (c) to the activity of said first reporter gene and said second reporter gene measured in the absence of said test compound.

3. A method according to claim 1, wherein step (c) is performed before step (b).

4. A method according to claim 1, 2, or 3, wherein said second reporter gene is indicative of the activity of a human immunodeficiency viruse hepatitis A virus, hepatitis B virus, hepatitis C virus, herpes simplex virus, human herpes virus, human rhinovirus, picornavirus, influenza virus, rhabdovirus, or papilloma virus.

5. A method according to claim 1, 2, or 3, wherein said second reporter gene is indicative of the activity of Salmonella, Shigella, Yersinia, Mycobacterium, Listeria, Stapylococcus, Chlamydiae, Legionella, or Leishmania.

6. A method according to claim 1, 2, or 3, wherein said first reporter comprises a humanized Renilla luciferase nucleic acid molecule that encodes a functional luciferase polypeptide, wherein said nucleic acid contains humanized codons not found in wild type Renilla reniformis luciferase, and wherein said humanized codons comprise at least 15% of the humanized codons shown in the nucleic acid sequence of SEQ ID NO:1.

7. A nucleic acid molecule encoding a functional Renilla luciferase polypeptide comprising a humanized Renilla luciferase nucleic acid molecule, wherein said nucleic acid contains humanized codons not found in wild type Renilla reniformis luciferase, and wherein said humanized codons comprise at least 15% of the humanized codons shown in the nucleic acid sequence of SEQ ID NO:1.

8. A nucleic acid according to claim 7, wherein said nucleic acid comprises a humanized Renilla luciferase nucleic acid molecule that encodes a functional luciferase polypeptide, wherein said nucleic acid contains humanized codons not found in wild type Renilla reniformis luciferase and said humanized codons comprise at least 25% of the humanized codons shown in the nucleic acid sequence of SEQ ID NO:1.

9. A nucleic acid molecule comprising the sequence of SEQ ID NO:1.

10. A construct comprising a vector and nucleic acid molecule according to claim 7.

11. A host cell comprising a nucleic acid molecule according to claim 7.

12. A host cell of claim 11, wherein said nucleic acid molecule comprises the sequence of SEQ ID NO:1.

13. A virus construct containing the nucleic acid molecule of claim 7.

14. A virus construct according to claim 13, wherein said virus is a human immunodeficiency virus, a hepatitis A virus, a hepatitis B virus, a hepatitis C virus, a herpes simplex virus, a human rhinovirus, a picornavirus, an influenza virus, a rhabdovirus, or a papilloma virus.

15. A kit comprising a nucleic acid molecule according to claim 7.


SEQUENCE LISTING (23 sequences)

SEQ ID NO: 1 - DNA, 936 bp, Artificial
  Renilla Luciferase Humanized Codons
atgacctccaaggtgtacgaccccgagcagcgcaagcgcatgattaccggcccccagtggtgggcccgctgcaagcagatgaacgtgctggacagcttcatcaactactacgacagcgagaagcacgccgagaacgccgtgatcttcctgcacggcaacgccgccagctcctacctgtggcgccacgtggtgcctcacatcgagcctgtggcccgctgcatcatccctgacctgatcggcatgggcaagagcggcaagagcggcaacggcagctaccgcctgctggaccactacaagtacctgaccgcctggttcgagctgctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggcctgacatcgaggaggacatcgccctgatcaagagcgaggagggcgagaagatggtgctggagaacaacttcttcgtggagaccatgctgcctagcaagatcatgcgcaagctggagcctgaggagttcgccgcctacctggagcccttcaaggagaagggcgaggtgcgccgccctaccctgagctggcctcgcgagatccctctggtgaagggcggcaagcctgacgtggtgcagatcgtgcgcaactacaacgcctacctgcgcgccagcgacgacctgcccaagatgttcatcgagagcgaccctggcttcttcagcaacgccatcgtggagggcgccaagaagttccctaacaccgagttcgtgaaggtgaagggcctgcacttcagccaggaggacgcccctgacgagatgggcaagtacatcaagagcttcgtggagcgcgtgctgaagaacgagcagtaa

SEQ ID NO: 2 - DNA, 936 bp, Artificial
  Renilla reniformis
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 3 - DNA, 75 bp, Artificial
  Oligonucleotide Template
atgacctccaaggtgtacgaccccgagcagcgcaagcgcatgattaccggcccccagtggtgggcccgctgcaag

SEQ ID NO: 4 - DNA, 38 bp, Artificial
  Oligonucleotide Primer
gaatcatctagaatgacctccaaggtgtacgaccccga

SEQ ID NO: 5 - DNA, 33 bp, Artificial
  Oligonucleotide Primer
gttcatgaattccttgcagcgggcccaccactg

SEQ ID NO: 6 - DNA, 99 bp, Artificial
  Oligonucleotide Template
gtgctggacagcttcatcaactactacgacagcgagaagcacgccgagaacgccgtgatcttcctgcacggcaacgccgccagctcctacctgtggcgc

SEQ ID NO: 7 - DNA, 100 bp, Artificial
  Oligonucleotide Primer
cgctcttgcccatgccgatcaggtcagggatgatgcagcgggccacaggctcgatgtgaggcaccacgtggcgccacaggtaggagctggcggcgttgcc

SEQ ID NO: 8 - DNA, 65 bp, Artificial
  Oligonucleotide Primer
gaatcatctagatgggcccgctgcaagcagatgaacgtgctggacagcttcatcaactactacga

SEQ ID NO: 9 - DNA, 70 bp, Artificial
  Oligonucleotide Primer
acttgtagtggtccagcaggcggtagctgccgttgccgctcttgccgctcttgcccatgccgatcaggtc

SEQ ID NO: 10 - DNA, 98 bp, Artificial
  Oligonucleotide Template
cgagctgctgaacctgcccaagaagatcatcttcgtgggccacgactggggcgcctgcctggccttccactacagctacgagcaccaggacaagatca

SEQ ID NO: 11 - DNA, 99 bp, Artificial
  Oligonucleotide template
cctcctcgatgtcaggccactcgtcccagctctcgatcacgtccaccacgctctcggcgtgcacgatggccttgatcttgtcctggtgctcgtagctgt

SEQ ID NO: 12 - DNA, 70 bp, Artificial
  Oligonucleotide Primer
accgcctgctggaccactacaagtacctgaccgcctggttcgagctgctgaacctgcccaagaagatcat

SEQ ID NO: 13 - DNA, 52 bp, Artificial
  Oligonucleotide Primer
catgatgaattctgatcagggcgatgtcctcctcgatgtcaggccactcgtc

SEQ ID NO: 14 - DNA, 97 bp, Artificial
  Oligonucleotide Template
gagaagatggtgctggagaacaacttcttcgtggagaccatgctgcctagcaagatcatgcgcaagctggagcctgaggagttcgccgcctacctgg

SEQ ID NO: 15 - DNA, 99 bp, Artificial
  Oligonucleotide Template
cttcaccagagggatctcgcgaggccagctcagggtagggcggcgcacctcgcccttctccttgaagggctccaggtaggcggcgaactcctcaggctc

SEQ ID NO: 16 - DNA, 62 bp, Artificial
  Oligonucleotide Primer
gatacatctagatgatcaagagcgaggagggcgagaagatggtgctggagaacaacttcttc

SEQ ID NO: 17 - DNA, 67 bp, Artificial
  Oligonucleotide Primer
agttgcgcacgatctgcaccacgtcaggcttgccgcccttcaccagagggatctcgcgaggccagct

SEQ ID NO: 18 - DNA, 100 bp, Artificial
  Oligonucleotide Template
cgcgccagcgacgacctgcccaagatgttcatcgagagcgaccctggcttcttcagcaacgccatcgtggagggcgccaagaagttccctaacaccgagt

SEQ ID NO: 19 - DNA, 100 bp, Artificial
  Oligonucleotide Template
agctcttgatgtacttgcccatctcgtcaggggcgtcctcctggctgaagtgcaggcccttcaccttcacgaactcggtgttagggaacttcttggcgcc

SEQ ID NO: 20 - DNA, 69 bp, Artificial
  Oligonucleotide Primer
cctgacgtggtgcagatcgtgcgcaactacaacgcctacctgcgcgccagcgacgacctgcccaagatg

SEQ ID NO: 21 - DNA, 72 bp, Artificial
  Oligonucleotide Primer
tcgatagaattcttactgctcgttcttcagcacgcgctccacgaagctcttgatgtacttgcccatctcgtc

SEQ ID NO: 22 - DNA, 22 bp, Artificial
  Primers used for Mutagenesis
cctctgtatcatatatgcttta

SEQ ID NO: 23 - DNA, 22 bp, Artificial
  Primers used for Mutagenesis
taaagcatatatgatacagagg